(19) United States
(12) Patent Application Publication
(10) Pub. No.: US 2024/0165202 A1
(43) Pub. Date: May 23, 2024

(54) USES OF FGF21 POLYPEPTIDES AND FUSION POLYPEPTIDES THEREOF

(71) Applicant: SUNSHINE LAKE PHARMA CO., LTD., Dongguan, Guangdong (CN)

(72) Inventors: Shushan LIN, Dongguan (CN); Yu LI, Dongguan (CN); Xianglei GAO, Dongguan (CN); Can XIE, Dongguan (CN); Jiangyu YAN, Dongguan (CN); Liang LIU, Dongguan (CN); Xiaoping LI, Dongguan (CN); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 18/282,619

(22) PCT Filed: Mar. 18, 2022
(86) PCT No.: PCT/CN2022/081572

§ 371 Date: Sep. 18, 2023

(30) Foreign Application Priority Data
Mar. 19, 2021 (CN) 202110296869.5

(51) Int. Cl. A61K 38/18; A61K 38/26; A61P 1/16

(52) U.S. Cl. CPC A61K 38/1825; A61K 38/26; A61P 1/16

(57) ABSTRACT

A method of treating fatty liver-related diseases in a patient includes administering to the patient a therapeutically effective amount of medicament manufactured from a FGF21 polypeptide, FGF21 fusion protein, or dual-fusion protein of FGF21 polypeptide and GLP-1 or a functional variant thereof.

### CROSS-REFERENCE TO RELATED APPLICATION

This application claims the priority and benefits of Chinese Patent Application No. 202110296869.5, filed with the State Intellectual Property Office of China on Mar. 19, 2021, which is incorporated herein by reference in its entirety.

### FIELD OF THE INVENTION

The present invention relates to the field of biomedicine, in particular, relates to the uses of FGF21 polypeptides and fusion polypeptides thereof.

### BACKGROUND ART

Glucagon-like peptide-1 (GLP-1) is an incretin secreted by small intestinal L cells, which can stimulate islet β cells to secrete insulin, thereby maintaining the balance of insulin level in patients. GLP-1 works indirectly through insulin, and only works on type 2 diabetes, which limits its use scope and effect; at the same time, it has been reported that GLP-1 has a potential risk of thyroid cancer.

FGF21 belongs to one of the members of the FGF family (fibroblast growth factors, FGFs). FGF21 can promote the absorption of glucose by adipocytes and enhance insulin sensitivity. And compared with insulin, FGF21 does not cause side effects such as hypoglycemia, and can more effectively protect β islet cells and promote the regeneration and repair of islet β cells. Furthermore, there is no potential tumor risk due to lack of mitogenic activity. FGF21 holds promise as a drug for the treatment of type 1 diabetes. In addition, FGF21 also has a good lipid-lowering effect and is a potential lipid-lowering drug. However, FGF21 also faces great challenges in druggability. On the one hand, due to the short half-life of FGF21, which is only about 1 h in mouse models (Xu et al., 2009). On the other hand, FGF21 has limited biological activity in vivo. Therefore, there is an urgent need to modify FGF21.

Chinese application CN111662373A discloses FGF21 polypeptides, fusion proteins, and fusion proteins comprise FGF21 polypeptides, Fc domains, and GLP-1 or functional variants thereof; it also discloses the use of these polypeptides and fusion proteins in the manufacture of a medicament for treating diseases caused by metabolic disorders of FGF21, the diseases comprise diabetes, fatty liver, obesity and/or pancreatitis, the experimental data of reducing fasting blood glucose, body weight, food intake and blood lipid are given in the examples.

NASH, namely nonalcoholic steatohepatitis, also known as metabolic steatohepatitis, is a clinical syndrome similar to alcoholic hepatitis in pathological changes but no history of excessive drinking. Its main feature is hepatocyte bullous steatosis with hepatocyte damage and inflammation, and severe cases can develop into liver fibrosis, liver cirrhosis, liver failure and liver tumors. Because patients do not show obvious symptoms in the early stage, it is called “silent killer”. Over the past 20 years, the incidence of its precursor nonalcoholic fatty liver disease (NAFLD) has doubled, and NASH becomes the leading cause of chronic liver disease and abnormal liver enzymes in the developed world. According to statistics, about 3-5% of people worldwide suffer from NASH, and according to an article published in Nature at 2017, NASH is about to become the leading cause of liver transplantation in the United States after chronic hepatitis C. Although NASH drugs have huge market prospects, and there are also many research institutions or pharmaceutical companies focusing on the research and development of NASH drugs, but in the past few decades, due to the very complex pathogenesis of NASH, scientists have encountered many setbacks and failures in the process of developing drugs, several blockbuster NASH drug candidates have ended in failure, and the world will not have the first drug on the market until 2020. Therefore, the research and development of NASH drugs has a long way to go.

### SUMMARY

The present application provides use of a FGF21 polypeptide or a fusion protein thereof in the manufacture of a medicament for treating fatty liver-related diseases.

In the first aspect, provided herein is use of a FGF21 polypeptide in the manufacture of a medicament for treating fatty liver-related diseases.

Alternatively, provided herein is a method of treating fatty liver-related diseases in a patient comprising administering to the patient a therapeutically amount of medicament manufactured from a FGF21 polypeptide.

Alternatively, provided herein is a medicament manufactured from a FGF21 polypeptide for use in treating fatty liver-related diseases in a patient.

In some embodiments, the FGF21 polypeptide has the amino acid sequence shown in SEQ ID NO: 1 or a variant thereof. Preferably, compared with the amino acid sequence shown in SEQ ID NO: 1, the FGF21 polypeptide comprises amino acid substitutions at the following positions: L98, S167 and P171. In the present application, the L98, S167 and P171 may respectively refer to the 98th residue L, the 167th residue S and the 171st residue P of the amino acid sequence shown in SEQ ID NO: 1.

Preferably, the FGF21 polypeptide further comprises amino acid substitutions at one or more positions selected from R175, R19, A180, A31 and G43. In the present application, the R175, R19, A180, A31 and G43 may respectively refer to the 175th residue R, the 19th residue R, the 180th residue A, the 31st residue A and the 43rd residue G of the amino acid sequence shown in SEQ ID NO: 1.

In some embodiments, compared with the amino acid sequence shown in SEQ ID NO: 1, the FGF21 polypeptide may comprise amino acid substitutions at the amino acid residue positions selected from:
(1) L98, S167, P171 and R175;
(2) L98, S167, P171, R175 and R19;
(3) L98, S167, P171, R175, R19 and A180;
(4) L98, S167, P171, R175, R19, A31 and G43.

Preferably, the amino acid substitution at L98 of the FGF21 polypeptide can be L98R;

preferably, the amino acid substitution at S167 of the FGF21 polypeptide can be S167H;

preferably, the amino acid substitution at P171 of the FGF21 polypeptide can be P171A or P171G;

preferably, the amino acid substitution at R175 of the FGF21 polypeptide can be R175L;

preferably, the amino acid substitution at R19 of the FGF21 polypeptide can be R19V;

preferably, the amino acid substitution at A31 of the FGF21 polypeptide can be A31C.

In some embodiments, compared with the amino acid sequence shown in SEQ ID NO: 1, the FGF21 polypeptide may comprise amino acid substitutions selected from: (1) L98R, S167H and P171A; (2) L98R, S167H, P171A and R175L; (3) L98R, S167H, P171A, R175L and R19V; (4) L98R, S167H, P171G, R175L and R19V; (5) L98R, S167H, P171G, R175L, R19V and A180E; (6) L98R, S167H, P171A, R175L, R19V and A180E; (7) L98R, S167H, P171A, R175L, R19V, A31C and G43C; (8) L98R, S167H, P171G, R175L, R19V, A31C and G43C.

In this application, the FGF21 polypeptide may comprise any one of the amino acid sequences shown in: SEQ ID NO: 2-7.

The medicament may also be a pharmaceutical composition, which may comprise a therapeutically effective amount of the FGF21 polypeptide, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

The FGF21 polypeptide can be used for the treatment of diseases caused by metabolic dysregulation of FGF21. The diseases caused by the metabolic dysregulation of FGF21 include diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

In the second aspect, provided herein is use of a FGF21 fusion protein in the manufacture of a medicament for treating fatty liver-related diseases.

Alternatively, provided herein is a method of treating fatty liver-related diseases in a patient comprising administering to the patient a therapeutically amount of medicament manufactured from a FGF21 fusion protein.

Alternatively, provided herein is a medicament manufactured from a FGF21 fusion protein for use in treating fatty liver-related diseases in a patient.

The FGF21 fusion protein comprises a FGF21 polypeptide and an Fc domain, wherein the FGF21 polypeptide is as described in the first aspect.

The FGF21 polypeptide is linked to the Fc domain by a linker to form a FGF21 fusion protein, which is also referred to as a single-target FGF21 fusion protein in this application.

In some embodiments, the immunoglobulin Fc domain is linked to the C-terminus of the FGF21 polypeptide. In some embodiments, the immunoglobulin Fc domain is the Fc of human IgG or a functional variant thereof. Preferably, the immunoglobulin Fc domain may be the Fc of human IgG (refer to the protein in UniProt KB or Swiss-Prot with accession number P01861.1). The Fc of human IgG may comprise the amino acid sequence shown in SEQ ID NO:8.

In some embodiments, the immunoglobulin Fc domain may also be a functional variant of the Fc of human IgG. For example, the functional variant of the Fc of human IgG may be a polypeptide or a protein obtained by modifying the amino acid sequence of the Fc of human IgG1 or IgG4 (preferably IgG4) at specific amino acid residues with natural or non-naturally occurring amino acids. For example, the modification can be made by inserting, replacing or deleting one or more conserved or non-conserved amino acids at specific positions, and can also include modification that introduce non-amino acid structures at specific positions.

In some embodiments, the functional variant of the immunoglobulin Fc domain is IgG-Fc-PAAK, which comprises the amino acid sequence shown in SEQ ID NO:9. The IgG-Fc-PAAK may comprise mutations of 5228P, F234A, L235A and/or R409K, and deletion of K447. That is, compared with the amino acid sequence shown in SEQ ID NO: 8, the 228th residue S of the IgG-Fc-PAAK is substituted with residue P, the 234th residue F is substituted with residue A, and the 235th residue L can be substituted with residue A, the 235th residue L can be substituted with residue A, and the 447th residue K can be deleted.

In some embodiments, the FGF21 fusion protein further comprises a linker connecting the FGF21 polypeptide to the Fc domain. Preferably, the linker is a peptide linker. Preferably, the N-terminus of the linker is linked to the C-terminus of the immunoglobulin Fc domain, and the C-terminus of the linker is linked to the N-terminus of the FGF21 polypeptide. Preferably, the linker comprises the amino acid sequence shown in SEQ ID NO:12.

Preferably, the single target fusion FGF21 protein has any one of amino acid sequences selected from SEQ ID NO: 13-18.

Preferably, the single target FGF21 fusion protein is any one of single target FGF21 fusion proteins selected from:
a single target FGF21 fusion protein 1 #, which comprises the amino acid sequence shown in SEQ ID NO: 13;
a single target FGF21 fusion protein 2 #, which comprises the amino acid sequence shown in SEQ ID NO: 14;
a single target FGF21 fusion protein 4 #, which comprises the amino acid sequence shown in SEQ ID NO: 15;
a single target FGF21 fusion protein 7 #, which comprises the amino acid sequence shown in SEQ ID NO: 16;
a single target FGF21 fusion protein 9 #, which comprises the amino acid sequence shown in SEQ ID NO: 17;
a single target FGF21 fusion protein 12 #, which comprises the amino acid sequence shown in SEQ ID NO: 18.

In some embodiments, the FGF21 fusion protein is a dimeric fusion protein. Preferably, the dimeric fusion protein is respectively two heavy chains IgG-Fc-PAAK, two linkers and two FGF21 polypeptides from the N-terminus to the C-terminus.

The single target FGF21 fusion proteins 1 #, 2 #, 4 #, 7 #, 9 #, 12 # that respectively comprise the amino acid sequences SEQ ID NO: 13-SEQ ID NO: 18 are amino acid sequences of a monomeric FGF21 polypeptide, a single linker and a single heavy chain Fc region.

The single target FGF21 fusion protein can be used for the treatment of diseases caused by metabolic dysregulation of FGF21. The diseases caused by the metabolic dysregulation of FGF21 include diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

The medicament can also be a pharmaceutical composition, which can comprise a therapeutically effective amount of the FGF21 fusion protein, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

In the third aspect, provided herein is use of a dual target fusion protein in the manufacture of a medicament for treating fatty liver-related diseases, wherein the dual target fusion protein comprises a FGF21 polypeptide and GLP-1 or a functional variant thereof.

Alternatively, provided herein is a method of treating fatty liver-related diseases in a patient comprising administering to the patient a therapeutically amount of medicament manufactured from a dual target fusion protein, wherein the dual target fusion protein comprises a FGF21 polypeptide and GLP-1 or a functional variant thereof.

Alternatively, provided herein is a medicament manufactured from a dual target fusion protein for use in treating fatty liver-related diseases in a patient, wherein the dual target fusion protein comprises a FGF21 polypeptide and GLP-1 or a functional variant thereof.

The fusion protein comprising a FGF21 polypeptide and GLP-1 or a functional variant thereof, which is also referred to as a dual target fusion protein in this application.

The fusion protein comprises the FGF21 polypeptide described in the first aspect and GLP-1 or a functional variant thereof.

In some embodiments, the GLP-1 or a functional variant thereof comprises any one of the amino acid sequences shown in SEQ ID NO: 10-11.

In some embodiments, the GLP-1 or a functional variant thereof is human GLP-1 (which has accession number POC6A0.1 in UniProt KB or Swiss-Prot). Preferably, the GLP-1 is a functional variant of human GLP-1. Preferably, the functional variant of human GLP-1 is GLP-1-GEG, which may comprise the amino acid sequence shown in SEQ ID NO:11. For example, the GLP-1-GEG may comprise mutations of A8G, G22E and R36G. Preferably, compared with the amino acid sequence shown in SEQ ID NO: 10, the 8th residue A of the GLP-1-GEG can be substituted with residue G, the 22nd residue G can be substituted with residue E, the 36th residue R can be substituted with residue G.

The fusion protein may also comprise an immunoglobulin Fc domain or a functional variant thereof.

In some embodiments, the immunoglobulin Fc domain is located between the FGF21 polypeptide and the GLP-1 or a functional variant thereof.

In some embodiments, the immunoglobulin Fc domain is linked to the N-terminus of the FGF21 polypeptide and to the C-terminus of the GLP-1 or a functional variant thereof; alternatively, the immunoglobulin Fc domain is linked to the C-terminus of the FGF21 polypeptide and to the N-terminus of the GLP-1 or a functional variant thereof.

In some embodiments, the immunoglobulin Fc domain is the Fc of human IgG or a functional variant thereof. Preferably, the immunoglobulin Fc domain may be the Fc of human IgG (refer to the protein in UniProt KB or Swiss-Prot with accession number P01861.1). The Fc of human IgG may comprise the amino acid sequence shown in SEQ ID NO:8.

In some embodiments, the immunoglobulin Fc domain may also be a functional variant of the Fc of human IgG. For example, the functional variant of the Fc of the human IgG may be a polypeptide or a protein obtained by modifying the amino acid sequence of the Fc of human IgG1 or IgG4 (preferably IgG4) at specific amino acid residues with natural or non-naturally occurring amino acids. For example, the modification can be made by inserting, replacing or deleting one or more conserved or non-conserved amino acids at specific positions, and can also include modification that introduce non-amino acid structures at specific positions.

In some embodiments, the functional variant of the immunoglobulin Fc domain is IgG-Fc-PAAK, which comprises the amino acid sequence shown in SEQ ID NO:9. The IgG-Fc-PAAK may comprise mutations of 5228P, F234A, L235A and/or R409K, and deletion of K447. That is, compared with the amino acid sequence shown in SEQ ID NO: 8, the 228th residue S of the IgG-Fc-PAAK is substituted with residue P, the 234th residue F is substituted with residue A, and the 235th residue L can be substituted with residue A, the 235th residue L can be substituted with residue A, and the 447th residue K can be deleted.

In some embodiments, the fusion protein further comprises a linker connecting the FGF21 polypeptide to the Fc domain or a functional variant thereof, and/or connecting GLP-1 or a functional variant thereof to the Fc domain or a functional variant thereof. Preferably, the linker is a peptide linker.

Preferably, the linker comprises a first linker and a second linker. Preferably, the first linker connects GLP-1 or a functional variant thereof to the Fc domain or a functional variant thereof, and the second linker connects the FGF21 polypeptide to the Fc domain or a functional variant thereof.

Preferably, the N-terminus of the first linker is linked to the C-terminus of GLP-1 or a functional variant thereof, and the C-terminus of the first linker is linked to the N-terminus of the Fc domain or a functional variant thereof; the C-terminus of the second linker is linked to the N-terminus of the FGF21 polypeptide, and the N-terminus of the second linker is linked to the C-terminus of the Fc domain or a functional variant thereof.

From the N-terminus to the C-terminus, the dual target fusion protein is respectively the GLP-1 or a functional variant thereof, the first linker, the immunoglobulin Fc domain, the second linker and the FGF21 polypeptide.

Alternatively, the C-terminus of the first linker is linked to the N-terminus of GLP-1 or a functional variant thereof, and the N-terminus of the first linker is linked to the C-terminus of the Fc domain or a functional variant thereof; the N-terminus of the second linker is linked to the C-terminus of the FGF21 polypeptide, and the C-terminus of the second linker is linked to the N-terminus of the Fc domain or a functional variant thereof.

From the N-terminus to the C-terminus, the dual target fusion protein is respectively the FGF21 polypeptide, the second linker, the immunoglobulin Fc domain, the first linker and the GLP-1 or a functional variant thereof.

Preferably, the first linker and/or the second linker comprise the amino acid sequence shown in SEQ ID NO:12.

Preferably, from the N-terminus to the C-terminus, the dual target fusion protein can be respectively the FGF21 polypeptide, the second linker, the immunoglobulin Fc domain, the first linker and the GLP-1 or a functional variant thereof, wherein the FGF21 polypeptide has any one of the amino acid sequences selected from SEQ ID NO: 2-7; the immunoglobulin Fc domain has any one of the amino acid sequences selected from SEQ ID NO: 8-9; the GLP-1 or a functional variant thereof has any one of the amino acid sequences selected from SEQ ID NO: 10-11; the first linker and/or the second linker has the amino acid sequence shown in SEQ ID NO:12.

Preferably, the dual target fusion protein is any one of the dual target fusion proteins selected from:
a dual target fusion protein 1 #, which comprises the amino acid sequence shown in SEQ ID NO: 19, from the N-terminus to the C-terminus, which is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO: 12) and FGF21-1 (comprising the amino acid sequence shown in SEQ ID NO: 2);
a dual target fusion protein 2 #, which comprises the amino acid sequence shown in SEQ ID NO: 20, from the N-terminus to the C-terminus, which is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO: 12) and FGF21-2 (comprising the amino acid sequence shown in SEQ ID NO: 3);
a dual target fusion protein 4 #, which comprises the amino acid sequence shown in SEQ ID NO: 21, from the N-terminus to the C-terminus, which is respectively GLP-1-GEG (comprising the amino acid sequence shown in SEQ ID NO: 11), the first linker (comprising the amino acid sequence shown in SEQ ID NO:12), IgG-Fc-PAAK (comprising the amino acid sequence shown in SEQ ID NO:9), the second linker (comprising the amino acid sequence shown in SEQ ID NO:12) and FGF21-4 (comprising the amino acid sequence shown in SEQ ID NO: 4);
a dual target fusion protein 7 #, which comprises the amino acid sequence shown in SEQ ID NO: 22, from the N-terminus to the C-terminus, which is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO:12) and FGF21-7 (comprising the amino acid sequence shown in SEQ ID NO: 5);
a dual target fusion protein 9 #, which comprises the amino acid sequence shown in SEQ ID NO: 23, from the N-terminus to the C-terminus, which is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO:12) and FGF21-9 (comprising the amino acid sequence shown in SEQ ID NO: 6);
a dual target fusion protein 12 #, which comprises the amino acid sequence shown in SEQ ID NO: 24, from the N-terminus to the C-terminus, which is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO:12) and FGF21-12 (comprising the amino acid sequence shown in SEQ ID NO: 7).

In some embodiments, the dual target fusion protein is a dimeric fusion protein. Preferably, the dimeric fusion protein is respectively two GLP-1-GEG, two first linkers, two heavy chains IgG-Fc-PAAK, two second linkers and two FGF21 polypeptides from the N-terminus to the C-terminus.

From the N-terminus to the C-terminus, the dual target fusion proteins 1 #, 2 #, 4 #, 7 #, 9 #, and 12 # which respectively comprise the amino acid sequences shown in SEQ ID NO: 19-SEQ ID NO: 24, are the amino acid sequences of a monomeric GLP-1-GEG, a first linker, a heavy chain IgG-Fc-PAAK, a second linker and a single FGF21 polypeptides, respectively.

For example, the dual target fusion protein 1 # is a dimer fusion protein, from the N-terminus to the C-terminus, which is respectively two GLP-1-GEG, two first linkers, two heavy chains IgG-Fc-PAAK, two second linkers and two FGF21 polypeptides, wherein a GLP-1-GEG, a first linker, a heavy chain IgG-Fc-PAAK, a second linker and a FGF21 have amino acid sequence shown in SEQ ID NO: 19, from the N-terminus to the C-terminus, the amino acid sequence is respectively GLP-1-GEG (comprising the amino acid sequence shown in SEQ ID NO: 11), the first linker (comprising the amino acid sequence shown in SEQ ID NO: 12), IgG-Fc-PAAK (comprising the amino acid sequence shown in SEQ ID NO: 9), the second linker (comprising the amino acid sequence shown in SEQ ID NO: 12) and FGF21-1 (comprising the amino acid sequence shown in SEQ ID NO: 2).

The medicament can also be a pharmaceutical composition, which can comprise a therapeutically effective amount of the dual target fusion protein, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

The dual target fusion protein can be used for the treatment of diseases caused by metabolic dysregulation of FGF21. The diseases caused by the metabolic dysregulation of FGF21 are diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

In the fourth aspect, provided herein is use of an isolated nucleic acid molecule in the manufacture of a medicament for treating fatty liver-related diseases, wherein the isolated nucleic acid molecule can encode the FGF21 polypeptide of the first aspect, the FGF21 fusion protein of the second aspect, or the dual fusion protein of the third aspect.

Alternatively, provided herein is a method of treating fatty liver-related diseases in a patient comprising administering to the patient a therapeutically amount of medicament manufactured from an isolated nucleic acid molecule, wherein the isolated nucleic acid molecule can encode the FGF21 polypeptide of the first aspect, the FGF21 fusion protein of the second aspect, or the dual fusion protein of the third aspect.

Alternatively, provided herein is a medicament manufactured from an isolated nucleic acid molecule for use in treating fatty liver-related diseases in a patient, wherein the isolated nucleic acid molecule can encode the FGF21 polypeptide of the first aspect, the FGF21 fusion protein of the second aspect, or the dual fusion protein of the third aspect.

The medicament can also be a pharmaceutical composition, which can comprise a therapeutically effective amount of the dual target fusion protein, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

The dual target fusion protein can be used for the treatment of diseases caused by metabolic dysregulation of FGF21. The diseases caused by the metabolic dysregulation of FGF21 are diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

In the fifth aspect, provided herein is use of a vector in the manufacture of a medicament for treating fatty liver-related diseases, wherein the vector comprises the isolated nucleic acid molecule of the fourth aspect.

Alternatively, provided herein is a method of treating fatty liver-related diseases in a patient comprising administering to the patient a therapeutically amount of medicament manufactured from a vector, wherein the vector comprises the isolated nucleic acid molecule of the fourth aspect.

Alternatively, provided herein is a medicament manufactured from a vector for use in treating fatty liver-related diseases in a patient, wherein the vector comprises the isolated nucleic acid molecule of the fourth aspect.

The vector may be a plasmid, cosmid, virus, phage or other commonly used vectors, such as used in the genetic engineering. For example, the vector is an expression vector.

The medicament can also be a pharmaceutical composition, which can comprise a therapeutically effective amount of the dual target fusion protein, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

The dual target fusion protein can be used for the treatment of diseases caused by metabolic dysregulation of FGF21. The diseases caused by the metabolic dysregulation of FGF21 are diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

In the sixth aspect, provided herein is use of a host cell in the manufacture of a medicament for treating fatty liver-related diseases, wherein the host cell may comprise or express the FGF21 polypeptide of the first aspect, or the FGF21 fusion protein of the second aspect, or the dual target fusion protein of the third aspect, or the isolated nucleic acid molecule of the fourth aspect, or the vector of the fifth aspect.

Alternatively, provided herein is a method of treating fatty liver-related diseases in a patient comprising administering to the patient a therapeutically amount of medicament manufactured from a host cell, wherein the host cell may comprise or express the FGF21 polypeptide of the first aspect, or the FGF21 fusion protein of the second aspect, or the dual target fusion protein of the third aspect, or the isolated nucleic acid molecule of the fourth aspect, or the vector of the fifth aspect.

Alternatively, provided herein is a medicament manufactured from a host cell for use in treating fatty liver-related diseases in a patient, wherein the host cell may comprise or express the FGF21 polypeptide of the first aspect, or the FGF21 fusion protein of the second aspect, or the dual target fusion protein of the third aspect, or the isolated nucleic acid molecule of the fourth aspect, or the vector of the fifth aspect.

The medicament can also be a pharmaceutical composition, which can comprise a therapeutically effective amount of the dual target fusion protein, and optionally pharmaceutically acceptable adjuvants. The pharmaceutically acceptable adjuvants may include buffers, antioxidants, preservatives, low molecular weight polypeptides, proteins, hydrophilic polymers, amino acids, sugars, chelating agents, counterions, metal complexes and/or non-ionic surfaces active agents, etc.

The pharmaceutical composition may be formulated for oral administration, intravenous administration, intramuscular administration, in situ administration at the tumor site, inhalation, rectal administration, vaginal administration, transdermal administration or subcutaneous reservoir administration.

The dual target fusion protein can be used for the treatment of diseases caused by metabolic dysregulation of FGF21. The diseases caused by the metabolic dysregulation of FGF21 are diabetes, fatty liver, obesity and/or pancreatitis. Preferably, the FGF21 polypeptide can be used for the treatment of fatty liver-related diseases, and the fatty liver-related diseases are non-alcoholic fatty liver disease (NAFLD), non-alcoholic steatohepatitis (NASH), liver fibrosis or cirrhosis. Preferably, the fatty liver-related disease is non-alcoholic steatohepatitis (NASH).

In the past 20 years, as obesity has become prevalent worldwide, and more and more people who are neither alcoholic nor viral hepatitis suffer from advanced liver disease, NASH has gradually gained attention. The “silent disease” of NASH is actually very scary, if not treated in time, it will cause liver fibrosis, which may eventually develop into liver cirrhosis, liver failure and even liver cancer. Patients have no choice but to get a liver transplant. After 2020, NASH will be the leading cause of liver transplants in the United States. However, there are only 1 or 2 medicaments approved for the treatment of NASH in the world because the incidence of NASH is very complicated. Some scholars proposed theories such as “second hit” and “multiple hits”, but so far, the specific pathogenesis of NASH is unknown.

The application found and confirmed that the fusion protein provided herein can not only exert lasting hypoglycemic and weight loss effects, but also improve blood lipids and liver function, specifically, significantly reduce liver function indicators: the levels of ALT (alanine aminotransferase) and AST (aspartate aminotransferase), and can reduce the levels of total cholesterol (TC) and triglyceride (TG) in the liver. In addition, the fusion protein provided herein can also significantly reduce liver tissue steatosis, inflammatory infiltration, liver ballooning and NAS scores, especially, significantly reduce liver fibrosis scores. These indicators show that the fusion protein herein can significantly improve the indicator of NASH, these remarkable effects are completely unexpected based on the contents disclosed in the prior art.

### Definition

In the application, the terms “protein” and “polypeptide” are interchangeable, and in their broadest sense, the terms refer to compounds composed of two or more amino acids, amino acid analogue, or peptidomimetic subunit.

In the application, the term “composition” generally refers to a combination of two or more substances, e.g., a combination of an active agent and other inert or active compounds.

In the application, the term “therapeutically effective amount” generally refers to the minimum dose of active ingredient required to produce a therapeutic benefit to a subject. For example, for the patients exhibiting or predisposing to type 2 diabetes, obesity, metabolic syndrome, or for preventing the onset thereof, the “therapeutically effective amount” refers to an amount capable of inducing, ameliorating or causing amelioration of pathological symptoms, disease progression, or physiological conditions associated with or countered by the above-mentioned disorders.

In the application, the term “subject” or “patient” may be human or non-human animals, more specifically, companion animals (such as dogs, cats, etc.), farm animals (such as cattle, sheep, pigs, horses, etc.) or laboratory animals (such as rats, mice, guinea pigs, etc.).

In the application, the term “linker” generally refers to a functional structure that can connect two or more polypeptides through peptide bonds. In the application, the terms “linker” and “joint” are interchangeable. The linker can be used when forming the fusion protein of the present application. The linker can be composed of amino acids linked together by peptide bonds. The linker of the present application can be of any length or composition. In some embodiments, the linker may be composed of 1-20 amino acids linked by peptide bonds. For example, the amino acid can be selected from the 20 naturally occurring amino acids. In some embodiments, the amino acid can be selected from: glycine, serine, alanine, proline, asparagine, glutamine, and lysine. In some embodiments, the linker is composed of sterically unhindered multiple amino acids. For example, the sterically unhindered amino acids can be glycine and alanine. The linker can be a G-rich polypeptide, for example, it can be selected from (G)3-S (i.e. “GGGS”), (G)4-S (i.e. “GGGGS”) and (G)5-S (i.e. “GGGGGS”). In some embodiments, the linker may be GGGGSGGGGS, GGGGSGGGGSGGGGS or GGGGSGGGGSGGGGSA. Other suitable linkers may be GGGGGSGGGSGGGGS, GGGKGGGG, GGGNGSGG, GGGCGGGG and GPNGG etc. The linker described herein can also be a non-peptide linker. For example, alkyl linker can be used, such as —NH—(CH₂)_{S}-C(O)—, wherein S=2-20. These alkyl linkers can be further substituted with any unhindered group, which may include, but are not limited to, lower alkyl (e.g. C1-C₆), lower acyl, halogen (e.g. Cl, Br), CN, NH₂ or phenyl. Exemplary non-peptide linker can also be a polyethylene glycol linker, wherein the molecular weight of the linker can be 100-5000 kD, e.g., 100-500 kD.

In the application, the term “fusion protein” generally refers to a protein fusing by two or more proteins or polypeptides. In the application, the fusion protein may comprise the FGF21 polypeptide. The genes or nucleic acid molecules encoding the two or more proteins or polypeptides can be linked to each other to form a fusion gene or fused nucleic acid molecule, which can encode the fusion protein. The fusion protein can be artificially created by recombinant DNA technology used for biological research or therapy. In the application, the fusion protein may further comprise domain other than the FGF21 polypeptide. In the application, the fusion protein may further comprise a linker connecting the FGF21 polypeptide and the domain other than the FGF21 polypeptide, and/or other domains.

In the application, the term “immunoglobulin Fc domain” generally refers to a domain comprising the CH2 and CH3 constant region portions of an immunoglobulin (e.g., an antibody). For example, the immunoglobulin Fc domain can be a domain comprising the hinge region, CH2 and CH3 constant region portions of an immunoglobulin (e.g., an antibody). For example, the immunoglobulin can be a human immunoglobulin. For example, the immunoglobulin can be human IgG1.

In the application, the term “functional variant” generally refers to a protein or polypeptide that is substituted, deleted or added one or more amino acids on the basis of the amino acid sequence of the target protein (e.g. the FGF21 polypeptide, the fusion protein or immunoconjugate, the immunoglobulin Fc domain or the GLP-1), yet still retains at least one biological property of the target protein. In the application, the “more” of the “one or more” amino acid substitutions generally refers to a substitution of more than 1 amino acid. For example, 1-30, 1-20, 1-10, 1-5 amino acid substitutions, such as 1, 2, 3, 4, 5, 6, 7, 8, 9, 10, 11, 12, 13, 14, 15, 16, 17, 18, 19, 20, 11, 12, 13, 14, 15, 16, 17, 18, 19, 20, 21, 22, 23, 24, 25, 26, 27, 28, 29, or 30 amino acid substitutions. For example, the functional variant may comprise a protein or polypeptide having an amino acid change by at least 1, such as 1-30, 1-20, 1-10, also such as 1, 2, 3, 4 or 5 amino acid substitutions, deletions and/or additions. The functional variant may substantially retain the biological properties of the protein or the polypeptide prior to change (e.g., substitution, deletion or addition). For example, the functional variant may retain at least 60%, 70%, 80%, 90%, or 100% of the biological activity of the protein or polypeptide prior to the change. For example, the substitution can be a conservative substitution.

In the application, the functional variant may also be the homologue of target protein (e.g., the FGF21 polypeptide, the fusion protein or immunoconjugate, the immunoglobulin Fc domain, or the GLP-1). In the application, the homologue can be, for example, a protein or polypeptide having at least about 85% (e.g., at least about 85%, about 90%, about 91%, about 92%, about 93%, about 94%, about 95%, about 96%, about 97%, about 98%, about 99% or higher) sequence homology with the amino acid sequence of the target protein.

In the application, the homology generally refers to the similarity or relatedness between two or more sequences. “Percent sequence homology” can be calculated by comparing the two sequences to be compared in the comparison window, determining the number of positions where the same nucleic acid base (e.g., A, T, C, G, I) or the same amino acid residue (e.g., Ala, Pro, Ser, Thr, Gly, Val, Leu, Ile, Phe, Tyr, Trp, Lys, Arg, His, Asp, Glu, Asn, Gln, Cys and Met) exists in the two sequences to obtain the number of matching position, and dividing the number of matching position by the total number of position in the comparison window (i.e., the window size), and multiplying the result by 100 to obtain the percent sequence homology.

The “FGF21 polypeptide” described herein is a wild-type FGF21 in some embodiments and a FGF21 variant in other embodiments.

The “GLP-1” described herein is a wild-type GLP-1 in some embodiments or a GLP-1 variant in other embodiments.

The “FGF21 of dual-target fusion protein 1 #” described herein includes both the free FGF21+Fc after the dual-target fusion protein 1 # is metabolized in vivo, and the unmetabolized or intact dual-target fusion protein 1 #.

The “GLP-1 of dual-target fusion protein 1 #” described herein includes both the free GLP-1+Fc after the dual-target fusion protein 1 # is metabolized in vivo, and the unmetabolized or intact dual-target fusion protein 1 #.

### DESCRIPTION OF THE DRAWINGS

FIG. 1 shows the non-reduced SDS-PAGE detection chart of dual target fusion protein 1 #(dual target 1 # for short).

FIG. 2 shows the reduced SDS-PAGE detection chart of the dual target fusion protein 1 #(dual target 1 # for short).

FIG. 3 shows the mass spectrum of dual target fusion protein 1 #(dual target 1 # for short).

FIG. 4 shows average plasma drug concentration-time curve.

FIG. 5 shows the random blood glucose changes of C57BL/6 mice induced by high-fat diet and significantly lowers blood glucose after the first administration.

FIG. 6 shows the effect of drugs on intraperitoneal glucose tolerance IPGTT of C57BL/6 mice induced by high-fat diet.

FIG. 7 shows the fasting blood glucose, insulin levels and HOMA-IR of C57BL/6 mice induced by high-fat diet.

FIG. 8 shows the effect of drugs on the body weight change rate and body fat mass of C57BL/6 mice induced by high-fat diet.

FIG. 9 shows the serum lipid levels of C57BL/6 mice induced by high-fat diet.

FIG. 10 shows the serum liver function indicators ALT/AST levels of C57BL/6 mice induced by high-fat diet.

FIG. 11 shows the liver absolute weights of C57BL/6 mice induced by high-fat diet.

FIG. 12 shows the liver homogenate lipid levels of C57BL/6 mice induced by high-fat diet.

FIG. 13 shows the liver histopathology photos (400 um) of C57BL/6 mice induced by high-fat diet.

FIG. 14 shows the liver pathology scores of C57BL/6 mice induced by high-fat diet.

FIG. 15 shows the random blood glucose for a 6-week dosing experiment with dual target fusion protein 1 # in ob/ob mice.

FIG. 16 shows the body weight growth rate for a 6-week dosing experiment with dual target fusion protein 1 # in ob/ob mice.

FIG. 17 shows the serum TC/TG for a 6-week dosing experiment with dual target fusion protein 1 # in ob/ob mice.

FIG. 18 shows the liver wet weight and liver index for a 6-week dosing experiment with dual target fusion protein 1 # in ob/ob mice.

FIG. 19 shows the liver function indicators for a 6-week dosing experiment with dual target fusion protein 1 # in ob/ob mice.

FIG. 20 shows the liver lipids for a 6-week dosing experiment with dual target fusion protein 1 # in ob/ob mice.

FIG. 21 shows the liver pathology scores for a 6-week dosing experiment with dual target fusion protein 1 # in ob/ob mice.

### EXAMPLES

Described below are the preferred embodiments of the present invention, and the protection scope of the present invention is not limited to these embodiments. It should be pointed out that for those skilled in the art, some modifications and improvements made on the basis of the inventive concept of the present invention all belong to the protection scope of the present invention. The reagents used without the manufacturer's indication are conventional products that can be obtained from the market.

### Example 1 Construction of Expression Vector Plasmid-X2

Suzhou Jinweizhi Biotechnology Co., Ltd. was entrusted to synthesize the target gene, which encodes the dual target fusion protein 1 #(comprising the amino acid sequence shown in SEQ ID NO: 19). The target gene sequence and vector plasmid pXC17.4 were digested with endonucleases HindIII and EcoRI (TAKARA, Japan) at 37° C., and the digested product was purified and recovered using Gel Extraction Kit according to the manufacturer's instructions. The purified and recovered target gene was ligated with the vector by using DNA Ligation Kit Ver.2.1 (TAKARA, Japan) according to the manufacturer's instructions, and then treated at a constant temperature of 16° C. for 1 h to obtain recombinant expression plasmids.

The above recombinant expression plasmids were transformed into competent cells DH5a, and the cells were taken and coated on an ampicillin plate. The single clones on the plate were picked and cultured in 1 ml LB medium (peptone 10 g/L, yeast extract 5 g/L, sodium chloride 10 g/L and agar 2%, antibiotic content 100 μg/L), and then the plasmids were extracted and verified to be correct by the sequencing of Guangzhou Aiki Biotechnology Co., Ltd. A series of verified correct expression vectors were extracted using the Invitrogen plasmid large extraction Kit, then digested with restriction endonuclease Pvu I (TAKARA, Japan), then linearized, purified and recovered by ethanol precipitation, the obtained expression vectors were stored at −20° C. for later use.

### Example 2 Vector Transfection and Expression in Cells

After the CHO host cells were recovered and cultured with Cellvento CHO-200 medium (Merck), the cells were collected for transfection when the cell density was about 4.76×10⁶ cells/mL. The transfected cells were about 1×10⁷ cell, and the plasmids were about 40m, the transfection was carried out by electric shock method (Bio-Rad, Gene pulser Xcell). Cells were cultured in 20 mL of Cellvento CHO-200 medium after electric shock. On the second day of culture, cells were collected by centrifugation and resuspended in 20 mL of Cellvento CHO-200 medium supplemented with L-Methionine sulfoximine (Sigma-aldrich) to a final concentration of 50 μM. When the cell density was about 0.6×10⁶ cell/mL, the obtained mixed clones were passaged with Cellvento CHO-200 medium, and the passaged cell density was about 0.2×10⁶ cell/mL. When the cell viability was about 90%, the cell culture medium was collected.

### Example 3 Purification and Detection of Fusion Proteins

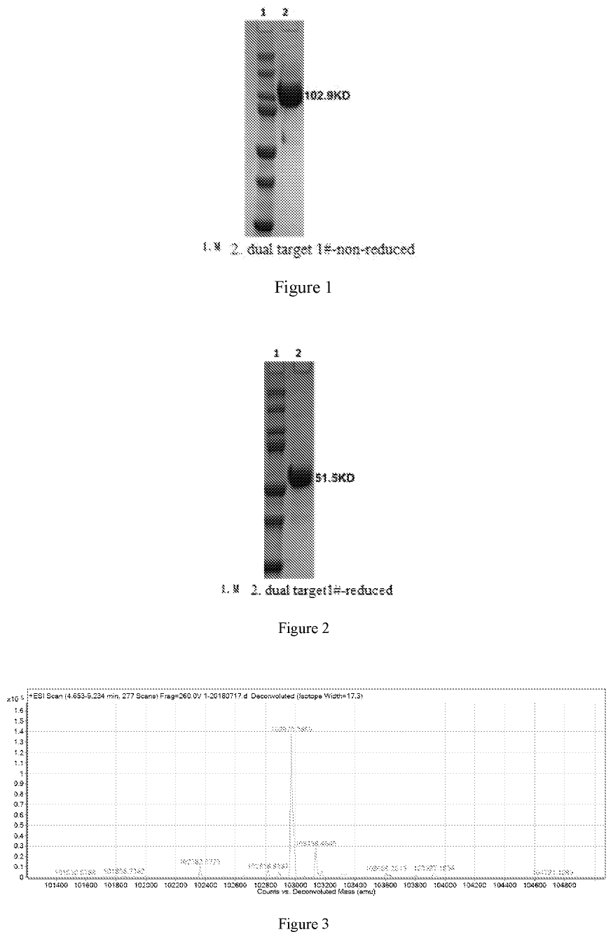

The cell culture medium was centrifuged at 200 g for 10 min, and the supernatant was centrifuged at 8000 rpm for 30 min and then collected. The centrifuged cell culture medium supernatant was affinity purified by protein A chromatography (EzFast Protein A Diamond, Borglon), the equilibrium solution was 20 mM PBS, 0.15M NaCl, pH7.4; the eluent was 0.1M glycine buffer with pH3.2. The protein eluate under the target absorption peak was collected, and after dialyzed against 20 mM PBS pH7.4 buffer, some samples were non-reduced (refer to FIG. 1) and reduced (refer to FIG. 2) and then detected by 10% SDS-PAGE electrophoresis. At the same time, some samples were taken for mass spectrometry detection, mass spectrometry (Accurate-Mass Q-TOF LC/MS, model G6530, Agilent Technologies) was used to detect the molecular weight, which was consistent with the theoretical molecular weight and was in the form of a homodimer (refer to FIG. 3).

### Example 4 Pharmacokinetics of Single Intravenous or Subcutaneous Injection in Male C57BL/6 Mice

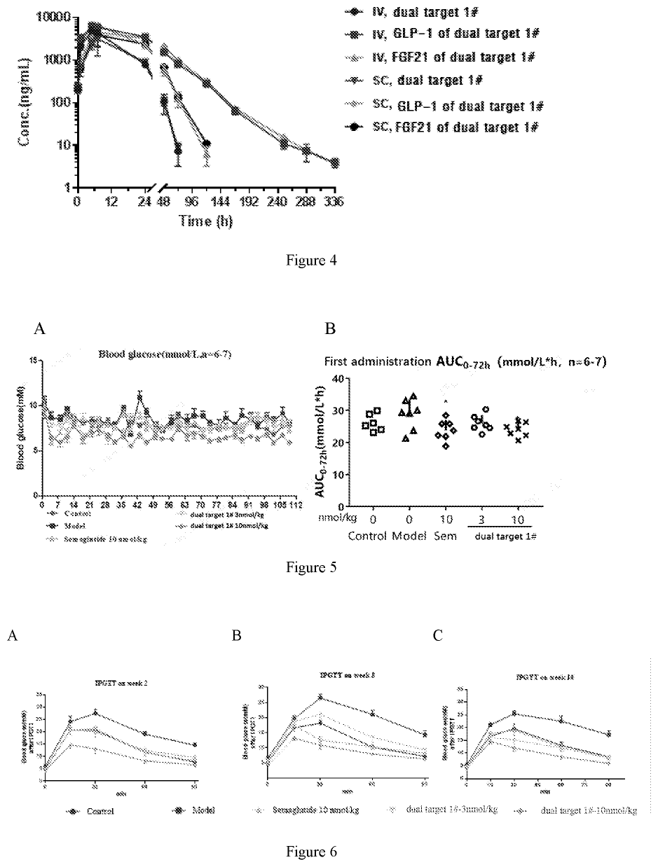

6 male C57BL/6 mice (Hunan SJA Laboratory Animal Co., Ltd.) were randomly divided into 2 groups, 3 mice/group, and the 2 groups were respectively administered the dual target fusion protein 1 # at 1 mg/kg intravenously or subcutaneously. Blood was collected at 1, 5, 7, 24, 48, 72, 120, 168, 240, and 336 h after administration, respectively, and plasma was separated (EDTA-K2 anticoagulation), in the intravenous administration group, blood was also collected at 0.25 h. The concentrations of the dual target fusion protein 1 #, FGF21 (Fc+FGF21) of the dual target fusion protein 1 # and GLP-1 (GLP-1+Fc) of the dual target fusion protein 1 # in each sample were analyzed by three ECLA methods (all based on the double-antibody sandwich principle). The pharmacokinetic parameters were calculated according to the plasma concentration, the main PK parameters are shown in Table 1, and the drug-time curve is shown in FIG. 4.

After subcutaneous injection of the dual target fusion protein 1 # in C57BL/6 mice, the average plasma half-life of the dual target fusion protein 1 #, FGF21 of the dual target fusion protein 1 # and GLP-1 of the dual target fusion protein 1 # were 7.2, 12 and 38 h, respectively; the average time to peak were 5.7, 7.0 and 6.3 h, and the average Cmax were 3.24, 3.96 and 4.45 μg/mL, the average AUCₗₐₛₜ were 60.7, 119 and 230 μg·h/mL, and the absolute bioavailability were 75%, 77% and 95%, respectively.

TABLE 1
Summary of main pharmacokinetic parameters after a single intravenous
or subcutaneous injection in C57BL/6 mice (average values)

| | | Adminis- | | | AUCₗₐₛₜ | | | | | Vz or | CL or | |
|---|---|---|---|---|---|---|---|---|---|---|---|---|
| | Test | tration | Dose | | μg · h/ | Cₘₐₓ | Tₘₐₓ | t_{1/2} | MRT | Vz/F | CL/F | F |
| Group | substance | way | mg/kg | Analyte | mL | μg/mL | h | h | h | mL/kg | mL/h/kg | % |
| 1 | Dual | i.v. | 1 | Dual target | 80.5 | 4.89 | 5.0* | 7.0 | 11 | 126 | 12.4 | / |
| | target | | | fusion | | | | | | | | |
| | fusion | | | protein 1# | | | | | | | | |
| | protein 1# | | | FGF21 of dual | 155 | 6.77 | 5.7* | 11 | 18 | 102 | 6.45 | / |
| | | | | target fusion | | | | | | | | |
| | | | | protein 1# | | | | | | | | |
| | | | | GLP-1 of dual | 243 | 6.50 | 5.0* | 39 | 37 | 228 | 4.11 | / |
| | | | | target fusion | | | | | | | | |
| | | | | protein 1# | | | | | | | | |
| 2 | Dual | s.c. | 1 | Dual target | 60.7 | 3.24 | 5.7 | 7.2 | 14 | 189 | 18.2 | 75 |
| | target | | | fusion | | | | | | | | |
| | fusion | | | protein 1# | | | | | | | | |
| | protein 1# | | | FGF21 of dual | 119 | 3.96 | 7.0 | 12 | 23 | 154 | 8.67 | 77 |
| | | | | target fusion | | | | | | | | |
| | | | | protein 1# | | | | | | | | |
| | | | | GLP-1 of dual | 230 | 4.45 | 6.3 | 38 | 43 | 237 | 4.37 | 95 |
| | | | | target fusion | | | | | | | | |
| | | | | protein 1# | | | | | | | | |

*During intravenous injection, part of the drug may be mistakenly injected into the subcutaneous tissue, resulting in an absorption peak. This intravenous administration datas are for reference only.

### Example 5 Dual Target Fusion Protein 1 # Exerts Lasting Hypoglycemic and Weight Loss Effects in High-Fat-Induced C57BL/6 Mice Model

After 16 weeks of high-fat diet feeding (D12492, Research Diets, Inc., New Brunswick, NJ), C57BL/6 mice (purchased from Hunan SJA Laboratory Animal Co., Ltd.) were randomly divided into vehicle group or treatment group according to the measured blood glucose and body weight. The study included control group with normal diet, high-fat diet Model group was administrated with vehicle PBS, subcutaneous injection of semaglutide once a day (QD) 10 nmol/kg, dual target 1 # was administrated twice weekly (BIW), the low dose was 3 nmol/kg and the high dose was 10 nmol/kg, 7 animals in each group. The drug groups were administrated the corresponding drugs according to the administration frequency for 16 consecutive weeks, and the high-fat diet was continued during the administration period until the end of the experiment. The random blood glucose, body weight and intraperitoneal glucose tolerance IPGTT of mice were monitored.

The random blood glucose of animals was monitored throughout the experimental period, and the results (see FIG. 5A) showed that blood glucose increased less in each group than in the Control group. The average blood glucose of the dual target fusion protein 1 # 10 nmol/kg was maintained at about 7.04 mmol/L during the administration, which was 22.4% lower than that of the model group and was the lowest among all groups, but the animals were in good conditions and did not experience hypoglycemia symptoms. After the first administration, compared with the Model group, both the positive drug semaglutide and the dual target fusion protein 1 # 10 nmol/kg could significantly reduce the blood glucose AUCO-72h (p<0.05) (refer to FIG. 5B).

The IPGTT experiment was performed on the 2nd (refer to FIG. 6A), 8th (refer to FIG. 6B), and 16th (refer to FIG. 6C) weeks, respectively. Compared with Control group, in FIG. 6, it can be seen that the intraperitoneal glucose tolerance (IPGTT) of the animals in Model group had significant increased in blood glucose before and after glucose administration, and the area under the blood glucose curve from 0 to 90 min also increased significantly, that is, the animals in Model group had abnormally high glucose tolerance. Compared with Model, both the dual target fusion protein 1 # and semaglutide could significantly reduce blood glucose at 30-90 min or 15-90 min after glucose administration, as well as the area under the blood glucose curve. The lowering effect of dual target fusion protein 1 # had a good dose-response effect, and the reduction of dual target fusion protein 1 # was more significant at an equimolar dose.

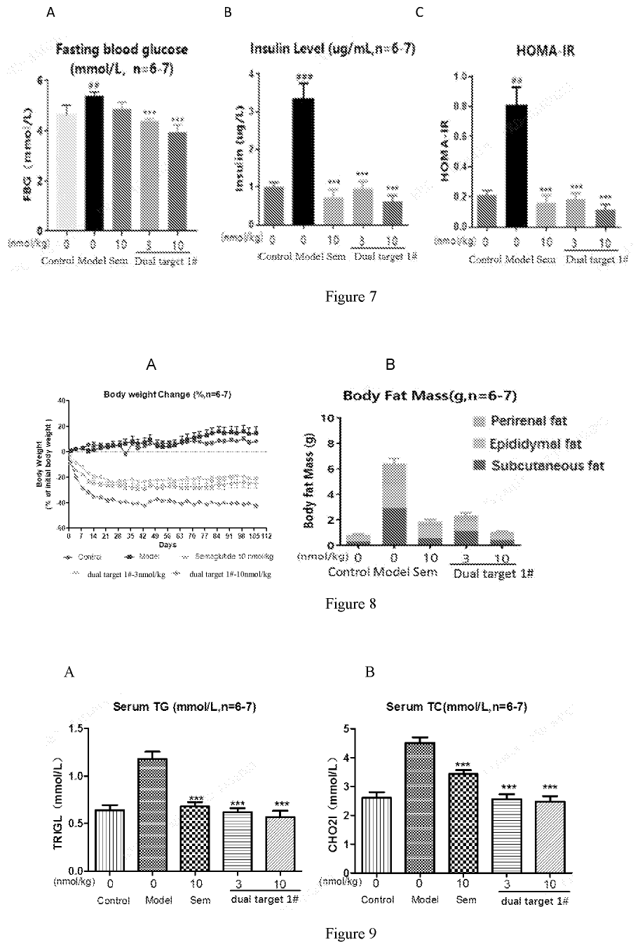

At the end of the experiment, the fasting blood glucose of animals were tested, and serum was taken to detect serum insulin levels and calculate the HOMA-IR index. The results in FIG. 7 show that: compared with the Control group, the fasting blood glucose (refer to FIG. 7A), insulin (refer to FIG. 7B) and HOMA-IR index (Homeostasis Model Insulin Resistance Index) (refer to FIG. 7C) of the animals in the Model group were significantly increased, that is, the animals in the model group showed insulin resistance. Compared with Model, both low and high doses of dual target fusion protein 1 # could significantly reduce fasting blood glucose levels (P<0.001), and the effect of reducing fasting blood glucose was better than that of semaglutide under equimolar dose conditions; both the dual target fusion protein 1 # and semaglutide in positive control group could significantly reduce insulin level and HOMA-IR index (P<0.001), and the dual target fusion protein 1 # had a significant dose-response effect in reduction, the dual target fusion protein 1 # reduced HOMA-IR as much as semaglutide under equimolar dose conditions.

During the administration period, the body weight of the animals was monitored and the body weight change rate was calculated. The results in FIG. 8 showed that the body weight of the Model group was much higher and the body weight continued to increase compared with the Control group. Compared with the Model group, both the dual target fusion protein 1 # and semaglutide could significantly reduce the body weight growth rate of animals (P<0.001), and the weight loss effect of the dual target fusion protein 1 # was significant, with a weight loss of 42.4% at a dose of 10 nmol/kg, while that of semaglutide at an equimolar dose was 24.3% (refer to FIG. 8A). The subcutaneous fat, epididymal fat and perirenal fat were taken from the animals during animal autopsy, and the total fat mass was calculated. The results in Table 2 showed that: compared with the Control, the body fat mass of Model was significantly increased, including subcutaneous fat, epididymal fat, perirenal fat and total fat. Compared with Model, both the dual target fusion protein 1 # and semaglutide in the positive control group could significantly reduce body fat mass (P<0.001), and the dual target fusion protein 1 # showed a significant dose-response relationship in reducing body fat, and the dual target fusion protein 1 # had a better reduction than semaglutide at an equimolar dose. (refer to FIG. 8B).

TABLE 2
Fat mass in C57BL/6 mice induced by high-fat diet (Mean ± SD, n = 6-7)

| | Dose | Subcutaneous | Epididymal | Perirenal | Total |
|---|---|---|---|---|---|
| Group | (nmol/kg) | fat (g) | fat (g) | fat (g) | Fat (g) |
| Control | 0 | 0.2 ± 0.1 | 0.4 ± 0.3 | 0.1 ± 0.1 | 0.6 ± 0.4 |
| Model | 0 | 2.9 ± 0.5^{###} | 1.8 ± 0.4^{###} | 1.7 ± 0.4^{###} | 6.4 ± 1.2^{###} |
| Semaglutide | 10 | 0.9 ± 1.0** | 1.0 ± 0.5** | 0.6 ± 0.6** | 2.6 ± 2.1** |
| Dual target | 3 | 1.1 ± 0.6*** | 0.7 ± 0.5*** | 0.5 ± 0.2*** | 2.3 ± 1.2*** |
| fusion protein 1# | | | | | |
| Dual target | 10 | 0.4 ± 0.1*** | 0.5 ± 0.2*** | 0.2 ± 0.0*** | 1.1 ± 0.3*** |
| fusion protein 1# | | | | | |

^{###}P < 0.001 vs Control; *, **, ***P < 0.05, 0.01, 0.001 vs Model.

### Example 6 Dual Target Proteins can Significantly Improve Blood Lipids and Liver Function in

At the end of the experiment of Example 4, that is, the 16th week of administration, the animals were fasted overnight and could drink water. After the animals were weighed, the orbital blood was collected, serum was separated, and serum lipids (TG (triglyceride), TC (total cholesterol)) and liver function indexes ALT (alanine aminotransferase)/AST (aspartate aminotransferase) were detected by an automatic biochemical analyzer. Serum insulin levels were detected by Elisa kit, and the HOMA-IR index was calculated according to the formula, that is, HOMA-IR=fasting blood glucose (blood glucose value at 0 time of IPGTT in the 16th week)×fasting insulin/22.5. After the blood was collected, the liver, subcutaneous fat, epididymal fat and perirenal fat were dissected and weighed. About 40-60 mg of liver tissue was weighed and put into a homogenization tube, 1 mL of absolute ethanol was added for homogenization, then the homogenate was taken and centrifuged at 4° C., 3,500 rpm for 10 min. The supernatant was taken, and the concentrations of TG and TC in the supernatant were detected by an automatic biochemical analyzer, and then the contents of TG and TC were calculated according to the weighed liver weight

FIG. 9 shows that, the levels of total cholesterol (TC) and triglyceride (TG) in Model animals were significantly increased compared with Control, which belonged to hyperlipidemia animal models. the dual target fusion protein 1 # and semaglutide in positive control group could significantly reduce serum TG compared with Model, and the dual target fusion protein 1 # had a significant dose-response effect in reduction effect. the dual target fusion protein 1 # reduced TG slightly better than semaglutide at an equimolar dose; the dual target fusion protein 1 # and semaglutide in positive control group could significantly reduce serum TC, and the dual target fusion protein 1 # had a significant dose-response effect in reduction effect, the dual target fusion protein 1 # reduced TC slightly better than semaglutide at an equimolar dose (refer to FIGS. 9A and 9B).

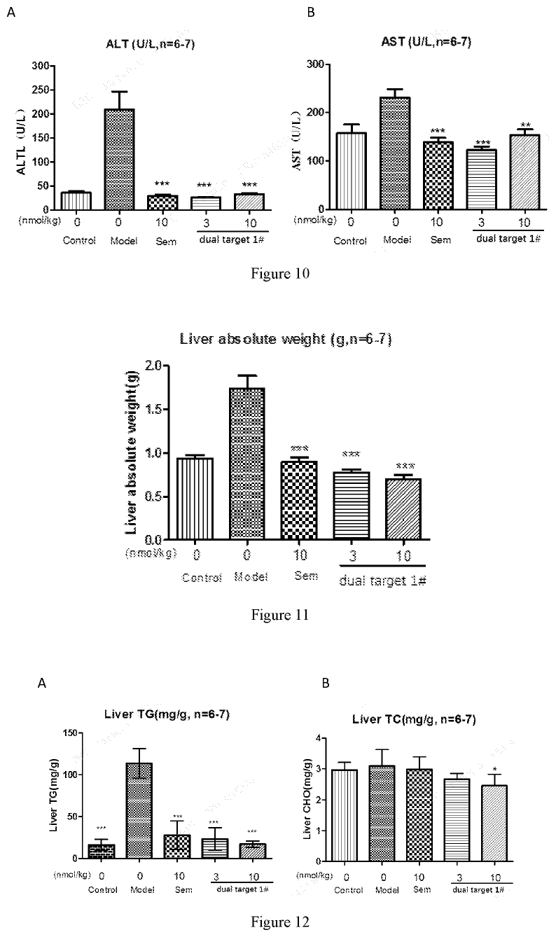

FIG. 10 shows that the serum liver function ALT and AST levels of the Model animals were significantly increased compared with the Control. The dual target fusion protein 1 # and semaglutide in positive control group could significantly reduce the levels of ALT and AST compared with the Model, and the reduction in the dual target fusion protein 1 # was comparable to that of semaglutide at an equimolar dose (refer to FIGS. 10A and 10B).

FIG. 11 shows that the liver absolute weight of the Model animals was significantly increased compared with Control. Compared with the Model, the dual target fusion protein 1 # and the semaglutide in positive control group could significantly reduce the liver absolute weight, and the dual target fusion protein 1 # had a significant dose-response effect in reduction effect. The reduction of the dual target fusion protein 1 # was slightly better than that of semaglutide at an equimolar dose (refer to FIG. 11).

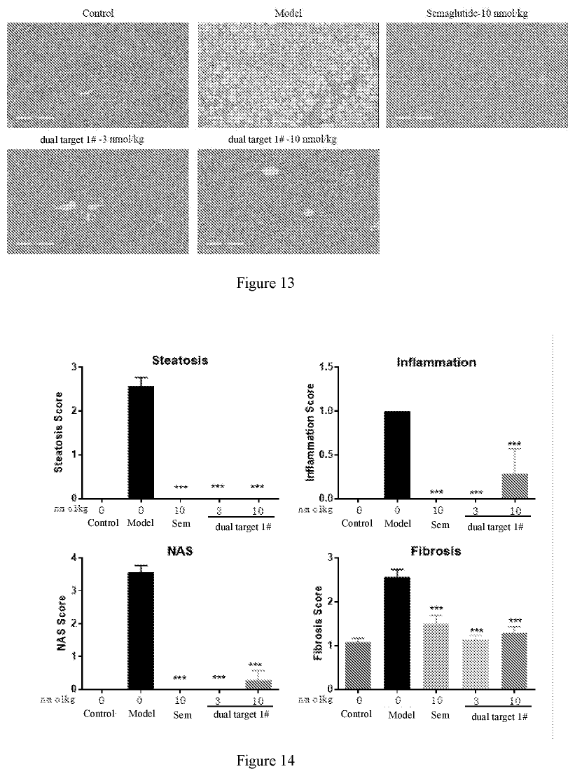

FIG. 12 shows that the levels of total cholesterol (TC) and triglyceride (TG) in liver homogenate of the Model animals were significantly increased compared with Control, which belonged to the hyperlipidemia animal models. The dual target fusion protein 1 # and semaglutide in positive control group could significantly reduce the level of TG in liver homogenate compared with the Model, and the dual target fusion protein 1 # has a significant dose-response effect in reduction effect. The dual target fusion protein 1 # reduced TG slightly better than semaglutide at an equimolar dose. The TC in liver homogenate of the Model was not significantly increased compared with Control, and there was no significant difference in the other groups except that the TC of the dual target fusion protein 1 #10 nmol/kg group was significantly lower than that of the model group (refer to FIGS. 12A and 12B).

### Example 7 Dual Target Proteins can Significantly Improve Liver NASH Indicators in High-Fat Diet Induced C57BL/6 Mice Model

The left lobe of the liver obtained by dissection in Example 5 was immersed in a 50 mL centrifuge tube filled with formalin to prepare liver disease sections, and the formalin liver tissue was subjected to wax block making, sectioning, HE staining, and HE staining slices were mailed to Suzhou KCl Co., Ltd. for NAS scoring; fibrosis was assessed by Sirius red staining, and the scores were scored according to the severity of fibrosis, with a score of 1-3. Liver histopathological changes were observed under light microscope and NAS/fibrosis scores were performed.

FIG. 13 shows that the liver tissue of the Model animals had severe fatty degeneration, vacuolization of hepatocytes, multiple inflammatory lesions, and mild to moderate fibrosis compared with the Control.

FIG. 14 and Table 3 show that, the dual target fusion protein 1 # and semaglutide in positive control group can significantly reduce liver steatosis, inflammatory infiltration and NAS scores compared with Model; in terms of fibrosis improvement, the dual target fusion protein 1 # and semaglutide in positive control group can significantly reduced liver fibrosis scores.

TABLE 3
NAS and fibrosis scores in liver tissue of high-fat diet
induced C57BL/6 mice (Mean ± SD, n = 6-7)

| | Dose | | | | | Fibrosis |
|---|---|---|---|---|---|---|
| | (nmol/ | Liver | Inflam- | Balloon | NAS total | Manual |
| Group | kg) | steatosis | mation | degeneration | score | Score |
| Control | 0 | 0.0 ± 0.0 | 0.0 ± 0.0 | 0.0 ± 0.0 | 0.0 ± 0.0 | 1.1 ± 0.2 |
| Model | 0 | 2.6 ± 0.5^{###} | 1.0 ± 0.0^{###} | 0.0 ± 0.0 | 3.6 ± 0.5^{###} | 2.6 ± 0.4^{###} |
| Semaglutide | 10 | 0.0 ± 0.0*** | 0.0 ± 0.0*** | 0.0 ± 0.0 | 0.0 ± 0.0*** | 1.5 ± 0.5** |
| Dual target fusion | 3 | 0.0 ± 0.0*** | 0.0 ± 0.0*** | 0.0 ± 0.0 | 0.0 ± 0.0*** | 1.1 ± 0.2*** |
| protein 1# | | | | | | |
| Dual target fusion | 10 | 0.0 ± 0.0*** | 0.3 ± 0.8* | 0.0 ± 0.0 | 0.3 ± 0.8*** | 1.3 ± 0.4*** |
| protein 1# | | | | | | |

##, ^{###}P < 0.01, 0.001 vs Control;
*, **, ***P < 0.05, 0.01, 0.001 vs Model.

### Example 8 the Effect of Lowering Blood Glucose after 6 Weeks of Administration in ob/ob Mice Model

70 Male ob/ob mice (Changzhou Cavens Laboratory Animal Co., Ltd.) weighed about 43-45 g were fed with high-fat diet (D12492, Research Diets, Inc., New Brunswick, NJ) for 9 weeks to establish a nonalcoholic steatohepatitis (NASH) model. 3 Weeks after HFD feeding, the therapeutic efficacy of the compounds dual target fusion protein 1 #, Fc-FGF21 (single target 1 #) and Dulaglutide were tested after 6 weeks of dosing. The experimental animals were randomly divided into 5 groups according to body weight and blood glucose after 2 weeks of HFD feeding: the model group (group-1, n=10), the dual target fusion protein 1 #-3 nmol/kg group (group-2, n=10); the dual target fusion protein 1 #-10 nmol/kg group (group-3, n=10 g); Fc-FGF21-10 nmol/kg group (group-4, n=10); Dulaglutide-10 nmol/kg (group-5, n=10). The model group was administrated PBS. The groups of dual target fusion protein 1 #, Fc-FGF21, and Dulaglutide were administrated twice a week for a total of 6 weeks. Feed intake, water intake and body weight of animals were recorded every two days after the start of HFD feeding until the end of the experiment. Blood glucose was measured at 0, 2, 7, 24, 48, 72 hours after the first administration, and then twice a week. At the end of the experiment, the animals were fasted to detect fasting blood glucose.

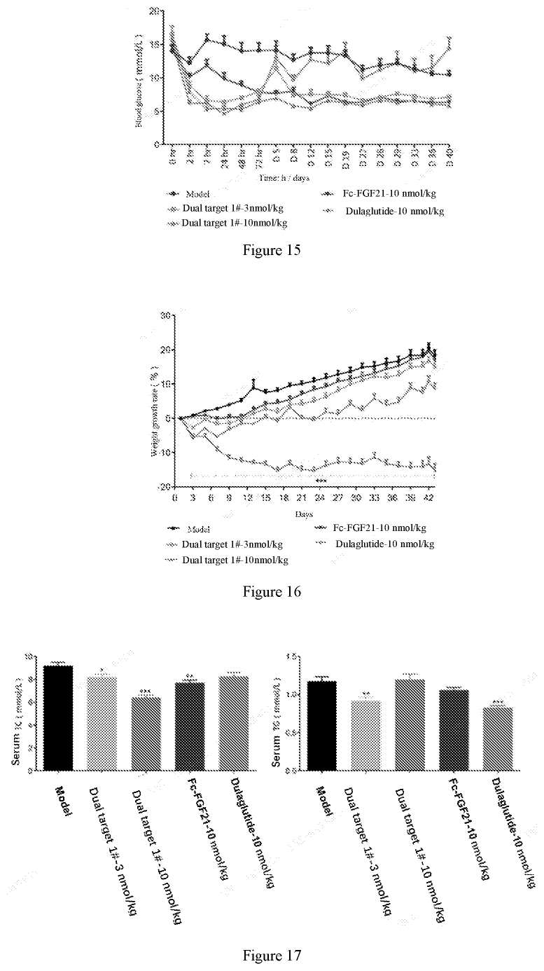

FIG. 15 shows that the blood glucose of the test compounds dual target fusion protein 1 # and Fc-FGF21 were lower than that of the model group from 7 h after administration to the whole experiment period from the random blood glucose level of long-term dosing. The dual target fusion protein 1 # showed a dose-dependent reduction in blood glucose, and the average hypoglycemic rates of low and high doses were 36.9% and 47.2%, respectively. After 5 days, the blood glucose level of the positive drug Dulaglutide was less different from that of the model group (P>0.05); at an equimolar dose, the hypoglycemic effect of dual target fusion protein 1 # was greater than that of Fc-FGF21 in the early stage of administration and greater than that of dulaglutide in the late stage of administration.

### Example 9 Administration of the Dual Target Fusion Protein 1 # for 6 Weeks can Significantly Reduce the Body Weight Change Rate of ob/ob Mice

The weight of animals in the model group continued to increase when they were given a high-fat diet until the end of the experiment, and the body weight growth rate was as high as 20.23%. FIG. 16 shows that the blood lipids in ob/ob mice were improved after administration of the dual target fusion protein 1 # for 6 weeks. Compared with the model group, the weight growth rate of the positive control Dulaglutide group and dual target fusion protein 1 # high dose group decreased significantly during the administration period from D1 to D43, and the average weight loss rates were respectively 8.9% and 22.7% (p<0.01 or 0.001); the body weight growth rate of animals with low dose of dual target fusion protein 1 # was significantly decreased (p<0.05 or 0.001) in the early stage of administration (day 1-day 37, D1-D37), and there was still a downward trend in the later stage of administration; the body weight growth rate of Fc-FGF21 group was decreased significantly (p<0.05-0.001) during the administration period from D5 to D21, and increased slowly in the later stage. The body weight growth rate of the dual target fusion protein 1 # at an equimolar dose was lower than that of the positive control Dulaglutide and Fc-FGF21.

### Example 10 Improvement of Blood Lipids in ob/ob Mice after 6 Weeks of Administration of the Dual Target Fusion Protein 1 #

FIG. 17 shows the improvement of blood lipids in ob/ob mice after 6 weeks of administration of dual target fusion protein 1 #. Compared with the model group, the serum TC levels of test compound dual target fusion protein 1 # in low dose or high dose, and Fc-FGF21 groups were all significantly decreased (p<0.05, p<0.001, p<0.01, respectively). And dual target fusion protein 1 # showed a good dose-dependence. The serum TC of positive control Dulaglutide group was not significantly decreased; at an equimolar dose, the reduction of TC by the dual target fusion protein 1 # was greater than that of dulaglutide and Fc-FGF21. The low dose group of dual target fusion protein 1 # and the positive control Dulaglutide group significantly decreased the level of serum TG (p<0.01, p<0.001); the high dose group of dual target fusion protein 1 # and Fc-FGF21 group did not significantly improve the level of serum TG.

### Example 11 Improvement of Liver Weight and NASH Indexes in ob/ob Mice of Administration of the Dual Target Fusion Protein 1 # for 6 Weeks

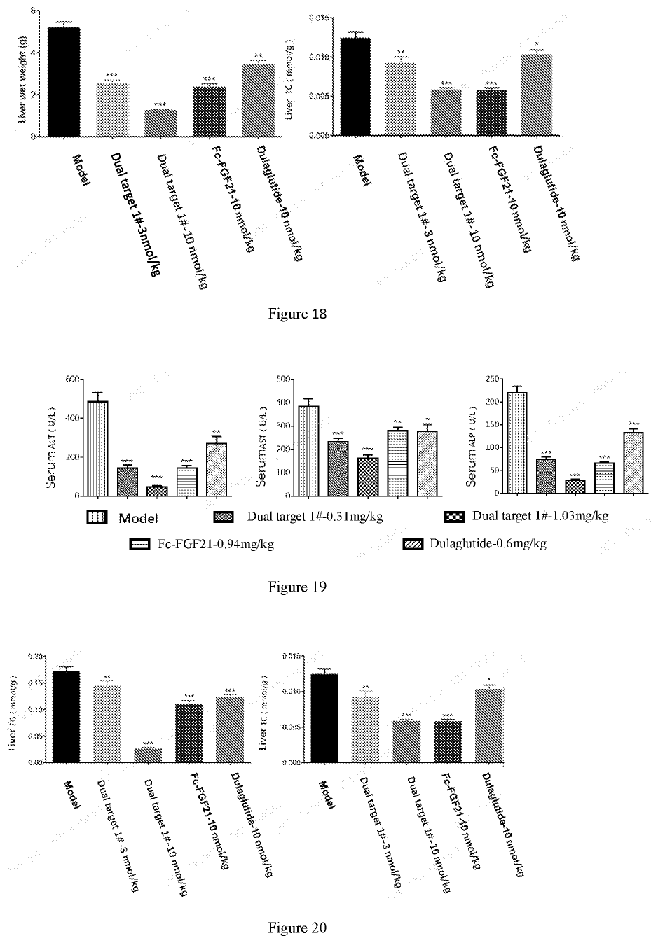

FIGS. 18-21 and Table 4 below show the improvement of liver weight and NASH indicators in ob/ob mice after administration of the dual target fusion protein 1 # for 6 weeks:

FIG. 18 shows the effect of the dual target fusion protein 1 # administration for 6 weeks on liver weight and liver index of ob/ob mice. Compared with the model group, the liver weight and liver index of animals in all groups were significantly decreased (p<0.001), and the dual target fusion protein 1 # group showed a good dose-dependence; at an equimolar dose, the reduction of liver weight and liver index of the dual target fusion protein 1 # was greater than that of Dulaglutide and Fc-FGF21;

FIG. 19 shows the effect of dual target fusion protein 1 # administration for 6 weeks on the serum ALT/AST/ALP levels of ob/ob mice. Compared with the model group, the serum ALT/AST/ALP levels of each administration group were significantly decreased (p<0.05, p<0.01, p<0.001), and the dual target fusion protein 1 # showed a good dose-dependence; at an equimolar dose, the reduction of ALT/AST/ALP of dual target fusion protein 1 # was greater than that of Dulaglutide and Fc-FGF21;

FIG. 20 shows the effect of the dual target fusion protein 1 # administration for 6 weeks on the content of TG and TC in the liver of ob/ob mice. Compared with the model group, the content of TG and TC in the liver of each administration group was significantly decreased (P<0.01˜P<0.05), and the dual target fusion protein 1 # showed a good dose-dependence; at an equimolar dose, dual target fusion protein 1 # reduced TG in liver more than Dulaglutide and Fc-FGF21, and reduced TC in liver more than Dulaglutide, which is comparable to Fc-FGF21;

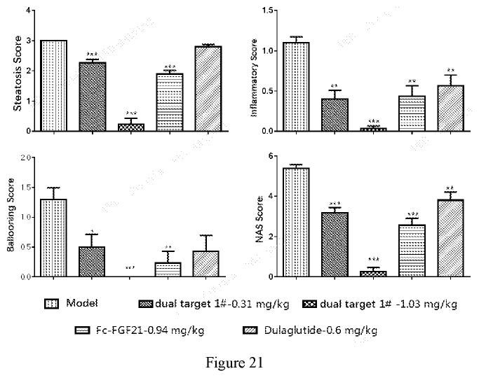

FIG. 21 shows the effect of the dual target fusion protein 1 # administration for 6 weeks on the liver pathological score of ob/ob mice. Compared with the Control group, the liver tissue of the Model group had severe fatty degeneration, and the hepatocytes were generally vacuolated, and inflammation occurred in many places, and mild to moderate fibrosis appeared. Compared with Model group, the high dose and low dose groups of dual target fusion protein 1 # showed a dose-dependent reduction in liver tissue steatosis, inflammatory infiltration, liver ballooning and NAS scores, and the reduction was greater than that of Dulaglutide and Fc-FGF21.

TABLE 4
Liver pathological scores in ob/ob mice of administration of the dual target fusion
protein 1# for 6 weeks (Mean ± SEM, n = 10)

| | | | Inflammatory | Liver | |
|---|---|---|---|---|---|
| | Dose | Steatosis | infiltration | ballooning | |
| Group | (nmol/kg) | Scores | Scores | Scores | NAS scores |
| Model group | 0 | 3.00 ± 0.00 | 1.10 ± 0.07 | 1.30 ± 0.20 | 5.40 ± 0.17 |
| Dual target | 3 | 2.27 ± 0.12*** | 0.40 ± 0.11** | 0.50 ± 0.21* | 3.17 ± 0.28*** |
| fusion protein 1# | | | | | |
| Dual target | 10 | 0.23 ± 0.20*** | 0.03 ± 0.03*** | 0.00 ± 0.00*** | 0.27 ± 0.20*** |
| fusion protein 1# | | | | | |
| Fc-FGF21 | 10 | 1.90 ± 0.11*** | 0.43 ± 0.13** | 0.23 ± 0.20** | 2.57 ± 0.34*** |
| Dulaglutide | 10 | 2.80 ± 0.07 | 0.57 ± 0.13** | 0.43 ± 0.26 | 3.80 ± 0.41** |

*, **, ***p < 0.05, 0.01, 0.001 vs. Model group

The foregoing detailed description has been presented by way of explanation and example, and is not intended to limit the scope of the claims. Various modifications to the embodiments presently enumerated in this application will be apparent to those of ordinary skill in the art and remain within the scope of the claims and equivalents.

1-26. (canceled)

27. A method of treating fatty liver-related diseases in a patient, the method comprising:
administering to the patient a therapeutically effective amount of medicament manufactured from a FGF21 polypeptide having the amino acid sequence shown in SEQ ID NO: 1 or a variant thereof.

28. The method of claim 27, wherein the FGF21 polypeptide comprises amino acid substitutions at the following positions: L98, S167 and P171.

29. The method of claim 27, wherein the FGF21 polypeptide comprises amino acid substitutions at one or more positions selected from R175, R19, A180, A31 and G43.

30. The method of claim 27, wherein compared with the amino acid sequence shown in SEQ ID NO: 1, the FGF21 polypeptide comprises amino acid substitutions at the amino acid residue positions selected from:
(1) L98, 5167, P171 and R175;
(2) L98, 5167, P171, R175 and R19;
(3) L98, 5167, P171, R175, R19 and A180; and
(4) L98, 5167, P171, R175, R19, A31 and G43.

31. The method of claim 27, wherein compared with the amino acid sequence shown in SEQ ID NO: 1, the FGF21 polypeptide comprises amino acid substitutions selected from:
(1) L98R, S167H and P171A;
(2) L98R, S167H, P171A and R175L;
(3) L98R, S167H, P171A, R175L and R19V;
(4) L98R, S167H, P171G, R175L and R19V;
(5) L98R, S167H, P171G, R175L, R19V and A180E;
(6) L98R, S167H, P171A, R175L, R19V and A180E;
(7) L98R, S167H, P171A, R175L, R19V, A31C and G43C;
(8) L98R, S167H, P171G, R175L, R19V, A31C and G43C;
(9) L98R, S167H, P171A, R175L, R19V, A31C and G43C; and
(10) L98R, S167H, P171G, R175L, R19V, A31C and G43C.

32. The method of claim 27, wherein the FGF21 polypeptide comprises any one of the amino acid sequences shown in SEQ ID NOs: 2-7.

33. A method of treating fatty liver-related diseases in a patient, the method comprising:
administering to the patient a therapeutically effective amount of medicament manufactured from a FGF21 fusion protein, wherein the FGF21 fusion protein comprises (i) a FGF21 polypeptide having the amino acid sequence shown in SEQ ID NO: 1 or a variant thereof, and (ii) an Fc domain.

34. The method of claim 33, wherein the immunoglobulin Fc domain is linked to the N-terminus of the FGF21; polypeptide.

35. The method of claim 33, wherein the FGF21 polypeptide is linked to the Fc domain by a linker.

36. The method of claim 33, wherein the FGF21 fusion protein comprises any one of amino acid sequences selected from SEQ ID NO: 13-18.

37-38. (canceled)

39. A method of treating fatty liver-related diseases in a patient, the method comprising:
administering to the patient a therapeutically effective amount of medicament manufactured from a dual target fusion protein, wherein the dual target fusion protein comprises (i) a FGF21 polypeptide having the amino acid sequence shown in SEQ ID NO: 1 and (ii) GLP-1 or a functional variant thereof.

40. The method of claim 39, wherein the GLP-1 or a functional variant thereof comprises the amino acid sequence shown in SEQ ID NO:10 or SEQ ID NO:11.

41. The method of claim 39, wherein the dual target fusion protein further comprises an immunoglobulin Fc domain or a functional variant thereof.

42. The method of claim 41, wherein the immunoglobulin Fc domain is located between the FGF21 polypeptide and the GLP-1 or a functional variant; thereof.

43. The method of claim 41, wherein the dual target fusion protein further comprises a linker connecting the FGF21 polypeptide to the Fc domain or a functional variant thereof, and/or connecting GLP-1 or a functional variant thereof to the Fc domain or a functional variant thereof.

44. The method of claim 43, wherein the linker comprises:
a first linker connecting GLP-1 or a functional variant thereof to the Fc domain or a functional variant thereof, and
a second linker connecting the FGF21 polypeptide to the Fc domain or a functional variant thereof; thereof.

45. The method of claim 43, wherein the dual target fusion protein comprises any one of the amino acid sequences selected from SEQ ID NOs: 19-24.

46. The method of claim 43, wherein the dual target fusion protein is a dimeric fusion protein.

47. The method of claim 45, wherein from the N-terminus to the C-terminus, any one of the amino acid sequences shown in SEQ ID NOs: 19-24 is the amino acid sequence of monomer GLP-1-GEG, a first linker, a heavy chain IgG-Fc-PAAK, a second linker and a FGF21 polypeptide, respectively.

48-78. (canceled)
